(19) United States
(12) Patent Application Publication
(10) Pub. No.: US 2026/0026947 A1
(43) Pub. Date: Jan. 29, 2026

(54) DRUG-ELUTING DEVICES, SYSTEMS, AND METHODS

(71) Applicant: Covidien LP, Mansfield, MA (US)

(72) Inventors: Danyong Zeng, Aliso Viejo, CA (US); Ravi Sadasivuni, Aliso Viejo, CA (US); Onnik E. Tchulluian, Costa Mesa, CA (US)

(21) Appl. No.: 19/275,362

(22) Filed: Jul. 21, 2025

Related U.S. Application Data
(60) Provisional application No. 63/676,138, filed on Jul. 26, 2024.

(51) Int. Cl. A61F 2/844; A61F 2/90

(52) U.S. Cl. CPC A61F 2/844; A61F 2/90

(57) ABSTRACT

Devices, systems, and methods for treating vascular defects are disclosed herein. According to some embodiments, the present technology includes an intravascular medical device comprising a tubular scaffold with a plurality of interconnected members. The tubular scaffold can be configured to expand from a collapsed state to an expanded state when implanted within a blood vessel. In some embodiments, the medical device further includes a plurality of carriers disposed about the tubular scaffold, each comprising a carrier body and a recess defined by the carrier body. A therapeutic agent may be released from the recesses in vivo, thereby providing a therapeutic effect to a treatment site.

### CROSS-REFERENCE TO RELATED APPLICATIONS

This application claims the benefit of and priority to U.S. Provisional Patent Application Ser. No. 63/676,138 filed Jul. 26, 2024, the entire disclosure of which is incorporated by reference herein.

### TECHNICAL FIELD

The present technology relates generally to devices and methods for treating blood vessels. Some embodiments of the present technology relate to drug-eluting devices for treating blood vessels.

### BACKGROUND

Intracranial atherosclerotic disease (ICAD) represents a significant cause of ischemic strokes, characterized by the narrowing of arteries within the brain due to the build-up of atherosclerotic plaques. This condition can lead to reduced blood flow, potentially resulting in severe neurological deficits or stroke. The complexity of ICAD is heightened by the intricate and delicate anatomy of intracranial vessels, which necessitates specialized interventions to restore adequate blood circulation. Traditional treatment options, including pharmacotherapy and invasive surgical procedures (e.g. bypass procedures), often face limitations due to the inaccessibility of intracranial arteries and the high risk of complications. Consequently, there is a need for minimally invasive and effective treatment modalities to address this condition.

The deployment of stents in intracranial arteries has emerged as a promising approach for treating ICAD, offering a minimally invasive solution to revascularize stenotic arteries and improve patient outcomes. However, existing stents face several challenges that can limit their efficacy and safety. These include issues with navigating the tortuous and narrow intracranial vessels, risks of in-stent restenosis, and potential complications such as stent thrombosis or vessel perforation. Additionally, the materials and designs of current stents may not always provide optimal flexibility and radial strength required for the unique environment of intracranial arteries. This is especially true when loaded drug-eluting markers are added to the stents.

In view of the above, there remains a need for improved devices and methods for treating ICAD.

### SUMMARY

The present technology relates generally to drug-eluting devices and methods for treating body lumens, including implantable drug-eluting devices. The subject technology is illustrated, for example, according to various aspects described below, including with reference to FIGS. 1A-4. Various examples of aspects of the subject technology are described as numbered clauses (1, 2, 3, etc.) for convenience. These are provided as examples and do not limit the subject technology.

1. An intravascular medical device, comprising:
a tubular scaffold comprising a plurality of interconnected members, the tubular scaffold being configured to expand from a collapsed state to an expanded state at a treatment site within a blood vessel; and
a plurality of carriers disposed about the tubular scaffold, each comprising a carrier body and a recess defined by the carrier body, wherein the recess contains a therapeutic agent, and wherein the carrier is configured to deliver the therapeutic agent to the treatment site.

2. The medical device of Example 1, wherein each of the plurality of carriers is mounted on one of the plurality of interconnected members.

3. The medical device of Example 2, wherein each of the carrier bodies comprises a tubular sidewall defining a lumen, and wherein each of the carrier bodies is mounted on a corresponding interconnected member such that the interconnected member is disposed within the lumen of the tubular sidewall.

4. The medical device of Example 3, wherein each of the recesses has a length less than a length of the carrier body and an arc length less than a circumference of the carrier body.

5. The medical device of Example 4, wherein the carriers are mounted on the interconnected members such that the recesses are disposed at an exterior surface of the tubular scaffold and such that the carrier is configured to release the therapeutic agent in a direction radially outward from the exterior surface.

6. The medical device of any one of Examples 2 to 5, wherein each of the carriers is disposed on the tubular scaffold such that a long axis of each of the carrier bodies is substantially parallel to a longitudinal axis of the tubular scaffold.

7. The medical device of any one of Examples 2 to 6, wherein each of the carrier bodies defines a slit extending along an entire length of the respective carrier body.

8. The medical device of any one of Examples 1 to 7, wherein each of the carrier bodies comprises an elongate strand disposed helically around the tubular scaffold.

9. The medical device of Example 8, wherein each of the recesses has a length less than a length of the carrier body and an arc length less than a perimeter of the carrier body.

10. The medical device of Example 9, wherein the carriers are disposed about the tubular scaffold such that the recesses are disposed at an exterior surface of the tubular scaffold and such that the carrier is configured to release the therapeutic agent in a direction radially from the exterior surface.

11. The medical device of any one of Examples 8 to 10, wherein each of the carriers are interwoven with the interconnected members such that a portion of each of the carriers is disposed within a lumen of the tubular scaffold and another portion of each of the carriers is disposed at an exterior surface of the tubular scaffold.

12. The medical device of any one of Examples 8 to 11, wherein each of the carriers is disposed around an exterior surface of the tubular scaffold.

13. The medical device of any one of Examples 8 to 12, wherein each of the carriers has a rectangular cross-sectional shape.

14. The medical device of any one of Examples 8 to 13, wherein each of the carriers has a circular cross-sectional shape.

15. The medical device of any one of Examples 1 to 14, wherein each of the carrier bodies comprise a polymer.

16. The medical device of any one of Examples 1 to 15, wherein each of the carrier bodies comprise a metal, wherein the metal comprises at least one of stainless steel, Nitinol, a platinum/iridium alloy, and/or Elgiloy.

17. The medical device of any one of Examples 1 to 16, wherein the carrier comprises at least one of platinum, silicone, Pebax®, or PTFE.

18. An intravascular medical device, the medical device comprising:
a tubular scaffold comprising a plurality of interconnected struts defining a plurality of open cells, wherein:
   the tubular scaffold is configured to transition between a delivery state and an expanded state,
   in the delivery state the tubular scaffold defines a first diameter around a longitudinal axis of the tubular scaffold, and
   in the expanded state the tubular scaffold defines a lumen and a second diameter around the longitudinal axis of the tubular scaffold, larger than the first diameter, and wherein the tubular scaffold is configured to be positioned at a treatment site within a blood vessel in the expanded state; and
a plurality of carriers disposed about the tubular scaffold, each comprising a carrier body and a recess defined by the carrier body, wherein the recess contains a therapeutic agent, and wherein the carrier is configured to deliver the therapeutic agent to the treatment site, and wherein the therapeutic agent is configured to reduce proliferation of vascular smooth muscle cells at the treatment site.

19. A method of treating a patient, comprising:
positioning a tubular scaffold at a treatment site within a blood vessel lumen, the tubular scaffold comprising a plurality of carriers disposed thereon, wherein each of the plurality of carriers comprises a carrier body and a recess defined by the carrier body, and wherein the recess contains a therapeutic agent; and
releasing the therapeutic agent to the treatment site.

20 The method of Example 19, wherein the tubular scaffold comprises a plurality of interconnected members, and wherein each of the plurality of carriers is mounted on one of the plurality of interconnected members.

21. The method of Example 20, wherein each of the carrier bodies comprises a tubular sidewall defining a lumen, and wherein each of the carrier bodies is mounted on a corresponding interconnected member such that the interconnected member is disposed within the lumen of the tubular sidewall.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a front view of a stent in accordance with several embodiments of the present technology.

FIG. 1B is an enlarged view of a portion of the stent of FIG. 1A in accordance with several embodiments of the present technology.

FIG. 2 is an isolated, perspective view of a carrier shown in FIGS. 1A and 1B in accordance with several embodiments of the present technology.

FIG. 3 is a front view of a stent in accordance with several embodiments of the present technology.

FIG. 4 is an isolated, perspective view of a portion of a carrier shown in FIG. 3 in accordance with several embodiments of the present technology.

### DETAILED DESCRIPTION

Excessive migration and proliferation of smooth muscle cells has been observed as a major factor contributing to the development of in-stent restenosis after vascular stenting. The smooth muscle cells form a neointimal layer within the stent that can narrow the vessel lumen and impede blood flow. To prevent smooth muscle cell proliferation, some stents include a drug-eluting coating. However, the coating is very thin (typically ranging from 5 μm to 15 μm) and is susceptible to scratching and detachment as it is advanced within a microcatheter and traverses the intricate anatomy of the brain. These issues can be more significant for self-expanding stents, as they remain in contact with the microcatheter during delivery due to their continuous outward radial force. In addition, vascular shear stress (i.e., the frictional force that flowing blood exerts on the endothelial surface of a blood vessel or stent) increases the risk of coating delamination post-implantation. Embodiments of the present technology address the foregoing limitations by providing a stent with discrete carriers mounted thereto, each carrier defining a recess containing a therapeutic agent. The recesses of the present technology reduce and/or eliminate unwanted shearing and/or damage during and after implantation of the stent, thereby increasing the efficacy of delivery of the drug to the treatment site once the stent is implanted.

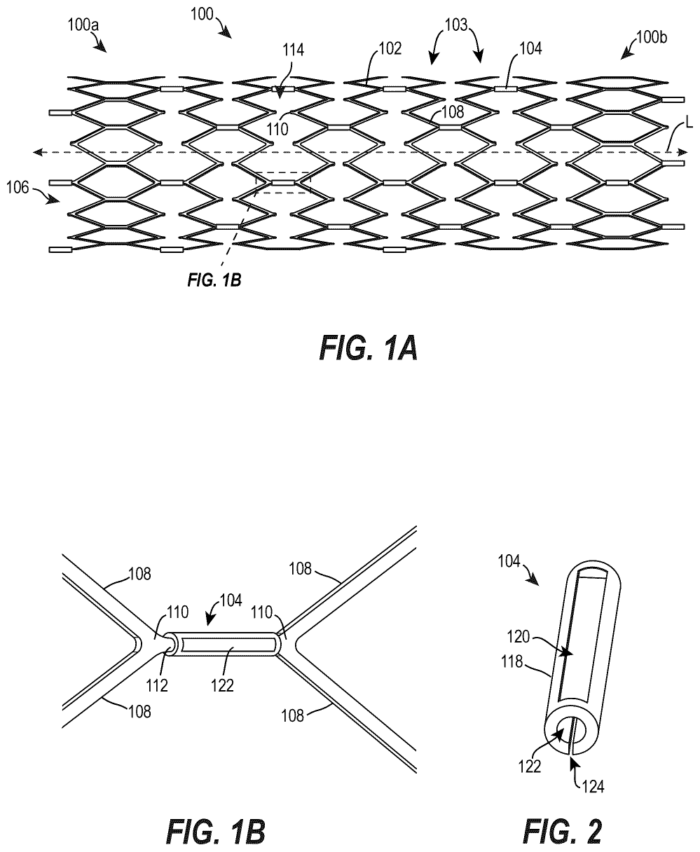

One such example of a medical device 100 (or “device 100”) configured in accordance with several embodiments of the present technology is shown in FIG. 1A. The device 100 may be configured for implantation within a diseased portion of an intracranial blood vessel, for example to treat atherosclerosis. As shown, the device 100 can comprise a tubular scaffold 102 and a plurality of carriers 104 mounted to the tubular scaffold 102. The device 100 has a first end portion 100a, a second end portion 100b, and a longitudinal axis L extending between the first and second end portions 100a, 100b. The device 100 further comprises a lumen 106 defined by an inner surface of the scaffold 102. The device 100 can have a low-profile delivery configuration (not shown) when constrained within a delivery catheter (e.g., a microcatheter) and an expanded configuration for engaging obstructions (such as atherosclerotic plaque) within the blood vessel lumen and/or for restoring blood flow within the blood vessel.

Referring still to FIG. 1A, the scaffold 102 can comprise a plurality of interconnected members that form the wall of the scaffold 102. In some examples, the interconnected members comprise a plurality of linear, zig-zagging struts 108 connected to one another at apices 110 by bridges 112. Together, the struts 110 and bridges 112 define and enclose a plurality of cells 114. The struts 108 may form a series of circumferential bands 103 (only a few labeled) disposed adjacent one another along a length of the scaffold 102, and the bridges 112 may extend longitudinally between adjacent bands 103. The bridges 112 may extend between every fourth pair of longitudinally opposing apices 110 (as shown in FIG. 1A), or may be distributed more or less frequently (e.g., a bridge 112 at every pair of apices, every other pair of apices 110, every third pair of apices 110, every fifth pair of apices 110, etc.) depending on the desired flexibility and/or surface coverage of the scaffold 102.

In some embodiments, the distal portion 100b of the device 100 can be generally tubular (e.g., cylindrical). In other embodiments, the device 100 can take any number of shapes or forms. The scaffold 102 can be formed of a superelastic material (e.g., Nitinol, a cobalt-chromium alloy, etc.) or other resilient material configured to self-expand when the scaffold 102 is released from a delivery catheter. In some embodiments, the tubular scaffold 102 has a substantially constant outer diameter (as shown), and in some embodiments, the tubular scaffold 102 has a varying outer diameter.

It will be appreciated that the tubular scaffold 102 can have other configurations. For example, the interconnected members (e.g., the struts and bridges) can have any suitable shape and be arranged in any suitable geometry (e.g., s-shaped and/or curved struts, curved and/or s-shaped bridges, open-cell design, closed-cell design, etc.). In some examples the tubular scaffold 102 may comprise a braid formed of one or more interwoven filaments. In other examples, the tubular scaffold 102 can comprise a coil.

The medical device 100 further comprises a plurality of carriers 104 mounted to the scaffold 102. In FIG. 1A, the carriers 104 are mounted only to the bridges 112, but in some embodiments may additionally or alternatively be mounted to the struts 108. Moreover, in some examples the carriers 104 may be disposed at the proximal and/or distal end portions 100a, 100b of the scaffold 102. In other embodiments, the carriers 104 are only mounted along the intermediate portion of the scaffold 102 (e.g., in between the proximal and distal end portions 100a, 100b) and not at the proximal and distal end portions 100a, 100b. In some embodiments, one or more of the carriers 104 are disposed on the tubular scaffold 102 such that a long axis of each carrier body 118 is substantially parallel to a longitudinal axis L of the tubular scaffold 102. In these and other embodiments, one or more of the carriers 104 are disposed on the tubular scaffold 102 such that a long axis of each carrier body 118 is substantially perpendicular to a longitudinal axis L of the tubular scaffold 102. It will be appreciated that the carriers 104 may be disposed on the tubular scaffold 102 at any angle or configuration relative to a longitudinal axis L of tubular scaffold 102.

FIG. 1B is an enlarged view of a portion of the medical device 100 including a carrier 104, and FIG. 2 is an enlarged view of a carrier 104 isolated from the tubular scaffold 102. Referring to FIGS. 1A-IC together, each of the carriers 104 can comprise a carrier body 118 and a recess 120 defined by an external surface the carrier body 118. The carrier body 118 can be generally tubular and define a lumen 122 configured to receive a portion of the tubular scaffold 102, such as one of the interconnected members, therein.

Each of the recesses 120 may contain a therapeutic agent 122 configured to be delivered to a treatment site within a bodily lumen. The therapeutic agent 122 is not shown in FIG. 2 for case of viewing the recess 120. In some embodiments, each recess 120 extends along only a portion of the length of the carrier body 118, and along only a portion of a circumference of the carrier body 118. Said another way, each recess 120 may have a length less than a length of the carrier body 118 and an arc length less than a circumference of the carrier body 118. These features may be advantageous in promoting directional release of the therapeutic agent 122. For example, one, some, or all of the carriers 104 can be mounted on the scaffold 102 such that the respective recesses 118 are facing away from the scaffold 102, toward the vessel wall. As such, the carriers 104 will release the therapeutic agent 120 in the direction of the vessel wall and reduce or eliminate release of the therapeutic agent 120 into the vessel lumen. The arc length of the recesses may be varied to achieve various release profiles. For instance, the individual recesses 118 may extend around no more than 15 degrees, 30 degrees, 45 degrees, 60 degrees, 75 degrees, 90 degrees, 135 degrees, or 180 degrees of the carrier body 118. In some embodiments, each recess 120 may span an entire length of the carrier body 118 and/or an entire circumference of the carrier body 118. In some embodiments, one, some, or all of the carriers 104 may have multiple discrete recesses 120 spaced apart along a length and/or circumference of the carrier body 118. In several of such embodiments, one, some, or all of the carriers 104 may be mounted such that at least one recess 120 is disposed at an exterior surface of the scaffold 102 and at least one recess 120 is disposed at an interior surface of the scaffold 102.

The carriers 104 may have the same or different shapes, the same or different lengths, the same or different recess geometries, and/or carry the same or different therapeutic agents. Moreover, the carriers 104 may be mounted to the scaffold 102 with their respective recesses 120 facing the same or different directions.

In some embodiments, the carrier 104 may comprise a slit 124 to facilitate attachment of the carrier 104 to the scaffold 102.

The carriers 104 may comprise various cross-sectional geometries. As shown in FIGS. 1A-1C, in some embodiments one, some, or all of the carriers 104 are substantially cylindrical and comprise a circular cross-sectional shape. In other embodiments, one, some, or all of the carriers 104 have a square cross-sectional shape, a rectangular cross-sectional shape, an elliptical cross-sectional shape, a hexagonal cross-sectional shape, a triangular cross-sectional shape, etc.). One, some, or all of the carriers 104 may comprise a metal or a metal alloy. In some embodiments, one, some, or all of the carriers 104 may comprise a polymer. In several examples, one, some, or all of the carriers 104 comprise at least one of platinum, silicon, or Pebax®.

The therapeutic agent 122 may be an anti-proliferative compound configured to prevent vascular smooth muscle growth around the medical device 100, thereby reducing neointimal hyperplasia, a post-interventional vascular condition characterized by the thickening of blood vessel walls and the gradual restenosis of the blood vessel lumen. The therapeutic agent 122 may additionally or alternatively be configured to reduce the risk of thrombosis at the treatment site. The therapeutic agent 122 may comprise one or more of an anti-proliferative compound, an immunosuppressant, a chemotherapy drug, an antincoplastic, or a combination thereof. In some embodiments, the therapeutic agent comprises sirolimus. In some embodiments, the therapeutic agent 122 comprises paclitaxel and/or zotarolimus. In some embodiments, a dual layer coating may be applied such that the dual layer coating comprises a first “shield” layer coating and a second layer coating of paclitaxel. In some embodiments, the first layer coating acts as an anti-thrombogenic agent.

It may be advantageous for the medical device 100 to deliver the therapeutic agent 122 to the treatment site in a sustained manner so as to provide continued resistance to smooth muscle cell proliferation, or extended treatment if used for another purpose. For example, the carriers 104 may release the therapeutic agent 122 at the treatment site over a time period of about at least three days, at least seven days, at least two weeks, at least one month, at least two months, at least three months, or longer. To facilitate the sustained release of the therapeutic agent 122 at the treatment site, in some embodiments, the therapeutic agent 122 may be combined with a biodegradable polymer (e.g., BioLinx™ polymer coating from Medtronic). In some embodiments, the polymer is disposed within the recesses 120 of the carriers 104.

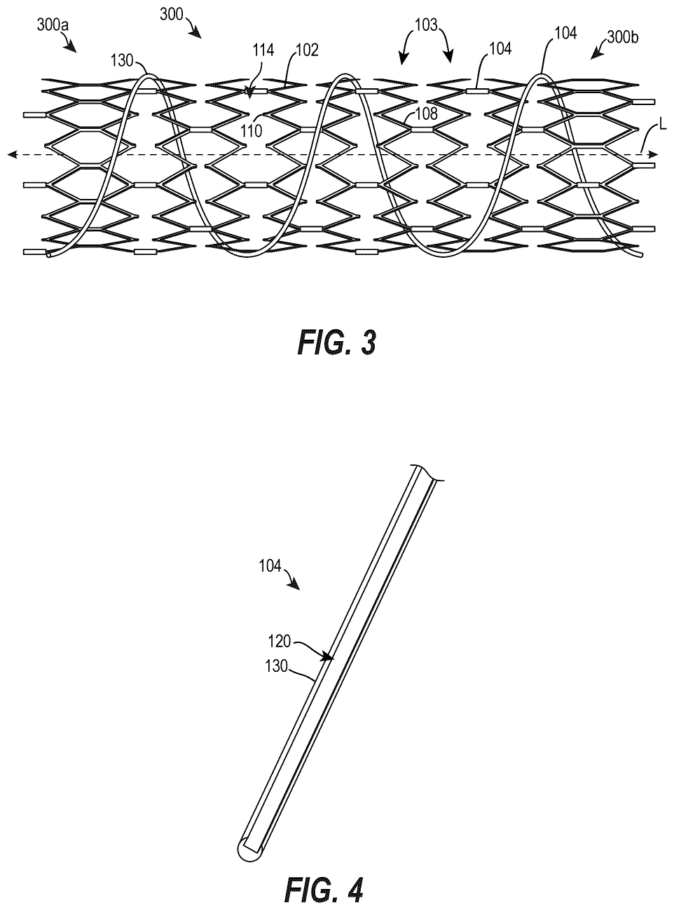

According to some aspects of the present technology, one or more of the carriers 104 may comprise an elongate strand. For example, FIG. 3 shows a medical device 300 comprising a tubular scaffold 102 and a carrier 104 in the form of an elongate strand 130 mounted helically around the scaffold 102. The tubular scaffold 102 can be generally similar to the tubular scaffold described above. FIG. 4 shows an isolated view of a portion of a carrier 104. While a single elongate strand 130 is shown in FIG. 3, the medical device 300 can include a plurality of elongate strands 130. Moreover, while the medical device 300 includes both an elongate carrier 104 and a plurality of shorter, cylindrical carriers 104, in other embodiments the medical device 300 may comprise only the elongate carrier(s) 104.

Each elongate strand 130 may comprise a recess 120 configured to deliver a therapeutic agent (not shown in FIG. 4) to a treatment site within a bodily lumen. In some embodiments, each recess 120 extends along only a portion of the length of the elongate strand 130, and along only a portion of a circumference of the elongate strand 130. Said another way, each recess 120 may have a length less than a length of the elongate strand 130 and an arc length less than a circumference of the elongate strand 130. These features may be advantageous in promoting directional release of the therapeutic agent. For example, the elongate strand 130 can be mounted on the scaffold 102 such that each recess 118 is facing away from the scaffold 102, toward the vessel wall. As such, the carrier(s) 104 will release the therapeutic agent 120 in the direction of the vessel wall and reduce or eliminate release of the therapeutic agent 120 into the vessel lumen. The arc length of the recess(es) 118 may be varied to achieve various release profiles. For instance, the recess(es) 118 may extend around no more than 15 degrees, 30 degrees, 45 degrees, 60 degrees, 75 degrees, 90 degrees, 135 degrees, or 180 degrees of the elongate strand 130. In some embodiments, each recess 120 may span an entire length of the elongate strand 130 and/or an entire circumference of the elongate strand 130.

The recess 120 may comprise a single recess 120 or a plurality of recesses 120 spaced apart along a longitudinal axis and/or circumference of the elongate strand 130. In several of such embodiments, the elongate strand(s) 130 may be mounted such that at least one recess 120 is disposed at an exterior surface of the scaffold 102 and at least one recess 120 is disposed at an interior surface of the scaffold 102.

As shown in FIGS. 3 and 4, the elongate strand 130 may be generally cylindrical and have a circular cross-sectional shape. In other embodiments, the elongate strand 130 can have a square cross-sectional shape, a rectangular cross-sectional shape, an elliptical cross-sectional shape, a hexagonal cross-sectional shape, a triangular cross-sectional shape, etc.). The elongate strand(s) 130 may comprise a metal, a metal alloy, or a polymer. In several examples, the elongate strand 130 may comprise at least one of platinum, silicon, or Pebax®.

In those embodiments in which multiple elongate strands 130 are used, the elongate strands 130 may have the same or different shapes, the same or different lengths, the same or different recess geometries, the same or different cross-sectional geometries, and/or carry the same or different therapeutic agents. Moreover, the elongate strands 130 may be mounted to the scaffold 102 with their respective recesses 120 facing the same or different directions.

While the embodiment represented in FIG. 3 shows the elongate strand 130 disposed helically around an exterior surface of the scaffold 102 and extends along substantially the entire length of the scaffold 102, the elongate strand(s) 130 could be coupled to any portion of the tubular scaffold 102 in any suitable configuration. For example, the elongate strand 130 may extend along only a portion of the length of the scaffold 102. In some cases, the elongate strand 130 may be mounted to an interior surface of the scaffold 102, and/or disposed helically around an inner surface of the scaffold 102. According to some examples, the elongate strand 130 may be interwoven through the cells 114 defined by the struts 108 and bridges 112 such that the elongate strand 130 is disposed at both the interior and exterior surfaces of the scaffold 102.

In some embodiments, the elongate strand(s) 130 is disposed along only a portion of the circumference of the scaffold 102. For example, the elongate strand 130 may be disposed in a sinusoidal shape and extend along the longitudinal axis L of the scaffold 102 with an amplitude such that two times the amplitude is less than a circumference of the scaffold 102. In other embodiments, the medical device can include one or more substantially linear elongate strands 130 disposed along the scaffold 102 such that a longitudinal axis of the linear carrier 104 is substantially parallel to a longitudinal axis L of the scaffold 102. In any of the foregoing examples, the elongate strand(s) 130 may be disposed only at the exterior of the scaffold 102 (and not the interior), only at the interior (and not the exterior), or both.

In any of the embodiments disclosed herein, the tubular scaffold 102 may comprise an outer coating in addition to the carriers 104. The coating may be disposed at all or a portion of the tubular scaffold 102. The outer coating may be configured to prevent thrombosis at the treatment site and/or increase the biocompatibility of the medical device. In some embodiments, the coating is an anti-thrombogenic coating. In some embodiments, the coating is configured to reduce platelet adhesion. In some embodiments, the coating is a synthetic coating. In some embodiments, the coating comprises phosphorylcholine.

In some embodiments, a method of treating a patient comprises delivering a medical device (such as medical device 100 or medical device 300) to a treatment site through an elongate delivery member (not shown) while in a low-profile, collapsed configuration for ease of navigation through a patient's vasculature. The treatment site can be at any location within the vasculature, including remote locations within the cerebral blood vessels, such as the intracranial artery (ICA) M1 and M2 segments as well as the P1 and P2 and A1, A2 and A3 segments of the vertebral and basilar arteries. When in position at the treatment site, the tubular scaffold 102 may be configured to self-expand into engagement with the blood vessel wall. Additionally or alternatively, the scaffold 102 may be expanded using one or more expansion means (e.g., an inflatable balloon, etc.). The carriers 104 may then release the therapeutic agent 122 at the treatment site.

Although many of the embodiments are described above with respect to devices, systems, and methods for treating ICAD and/or intracranial blood vessels, other applications and other embodiments in addition to those described herein are within the scope of the technology. For example, the stents and/or carriers of the present technology may be used to treat blood vessels outside of the brain (e.g., pulmonary blood vessels, blood vessels within the legs, etc.), and/or for treating multiple disease states.

### CONCLUSION

Although many of the embodiments are described above with respect to devices, systems, and methods for mechanical thrombectomy, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-4.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An intravascular medical device, comprising:
a tubular scaffold comprising a plurality of interconnected members, the tubular scaffold being configured to expand from a collapsed state to an expanded state at a treatment site within a blood vessel; and
a plurality of carriers disposed about the tubular scaffold, each comprising a carrier body and a recess defined by the carrier body, wherein the recess contains a therapeutic agent, and wherein the carrier is configured to deliver the therapeutic agent to the treatment site.

2. The medical device of claim 1, wherein each of the plurality of carriers is mounted on one of the plurality of interconnected members.

3. The medical device of claim 2, wherein each of the carrier bodies comprises a tubular sidewall defining a lumen, and wherein each of the carrier bodies is mounted on a corresponding interconnected member such that the interconnected member is disposed within the lumen of the tubular sidewall.

4. The medical device of claim 3, wherein each of the recesses has a length less than a length of the carrier body and an arc length less than a circumference of the carrier body.

5. The medical device of claim 4, wherein the carriers are mounted on the interconnected members such that the recesses are disposed at an exterior surface of the tubular scaffold and such that the carrier is configured to release the therapeutic agent in a direction radially outward from the exterior surface.

6. The medical device of claim 2, wherein each of the carriers is disposed on the tubular scaffold such that a long axis of each of the carrier bodies is substantially parallel to a longitudinal axis of the tubular scaffold.

7. The medical device of claim 2, wherein each of the carrier bodies defines a slit extending along an entire length of the respective carrier body.

8. The medical device of claim 1, wherein each of the carrier bodies comprises an elongate strand disposed helically around the tubular scaffold.

9. The medical device of claim 8, wherein each of the recesses has a length less than a length of the carrier body and an arc length less than a perimeter of the carrier body.

10. The medical device of claim 9, wherein the carriers are disposed about the tubular scaffold such that the recesses are disposed at an exterior surface of the tubular scaffold and such that the carrier is configured to release the therapeutic agent in a direction radially from the exterior surface.

11. The medical device of claim 8, wherein each of the carriers are interwoven with the interconnected members such that a portion of each of the carriers is disposed within a lumen of the tubular scaffold and another portion of each of the carriers is disposed at an exterior surface of the tubular scaffold.

12. The medical device of claim 8, wherein each of the carriers is disposed around an exterior surface of the tubular scaffold.

13. The medical device of claim 8, wherein each of the carriers has a rectangular cross-sectional shape.

14. The medical device of claim 8, wherein each of the carriers has a circular cross-sectional shape.

15. The medical device of claim 1, wherein each of the carrier bodies comprise a polymer.

16. The medical device of claim 1, wherein each of the carrier bodies comprise a metal, wherein the metal comprises at least one of stainless steel, Nitinol, a platinum/iridium alloy, and and/or Elgiloy.

17. The medical device of claim 1, wherein the carrier comprises at least one of platinum, silicone, Pebax®, or PTFE.
